(19) United States
(12) Patent Application Publication
(10) Pub. No.: US 2014/0037816 A1
(43) Pub. Date: Feb. 6, 2014

(54) STABILIZED MULTIPHASE AQUEOUS COMPOSITIONS

(71) Applicant: HERCULES INCORPORATED, Wilmington, DE (US)

(72) Inventors: Kirill N. Bakeev, Newark, DE (US); Brian John Huebner, Newark, DE (US); Gijsbert Kroon, Giessendam (NL); Tuttu Maria Nuutinen, Delft (NL)

(73) Assignee: HERCULES INCORPORATED, Wilmington, DE (US)

(21) Appl. No.: 13/951,944

(22) Filed: Jul. 26, 2013

Related U.S. Application Data
(60) Provisional application No. 61/677,582, filed on Jul. 31, 2012.

(51) Int. Cl. A23D 7/005; A61K 8/73; A61Q 19/00; C09D 7/12

(52) U.S. Cl. CPC A23D 7/005; C09D 7/125; A61K 8/731; A61Q 19/00
USPC 426/564; 106/162.8; 524/42; 510/109; 426/602; 426/654

(57) ABSTRACT

Aqueous multiphase compositions are stabilized using a blend of cellulose ether and nanocrystalline cellulose. The multiphase compositions can include particulates dispersed in water, oil in water emulsions, foams, and combinations of these systems. The blend can be used to stabilize aqueous paint systems, personal care products such as shampoos and detergents formed as oil in water emulsions, and foaming products such as detergents, shampoos, other personal care products and edible compositions such as whip cream substitutes. Further, this blend can reduce or eliminate the need for surfactants in many of these multiphase compositions.

### CROSS REFERENCE TO RELATED APPLICATIONS

The present application claims the benefit under 35 U.S.C. 119 (e) of U.S. Provisional Patent Application Ser. No. 61/677,582, filed Jul. 31, 2012, the entire content of which is hereby expressly incorporated herein by reference.

### BACKGROUND OF THE INVENTION

1. Field of the Disclosed and Claimed Inventive Concepts

The presently disclosed and claimed inventive concept(s) relates to multiphase systems including combinations of gas stabilized in liquid in the form of foams, solids suspended in liquids, as well as oil emulsified in water, or vice versa, in the form of an emulsion. Generally, particles will separate or settle from a liquid. However, the high viscosity of a liquid can help to maintain dispersed particles in suspension. Dispersants can also be used to maintain solids in suspension.

2. Background and Applicable Aspects of the Presently Disclosed and Claimed Inventive Concept(s)

Likewise, oil will generally tend to separate from water but can be stabilized using various aids, in particular, various surfactants. In many applications, the particular surfactants may be undesirable. For example, in certain applications, anionic surfactants can be irritating. With food products, it is desirable in many cases to minimize chemical additives. Foams will also break down quickly if not stable.

Cellulose ethers can be added to many of these multiphase systems to provide stabilization and, in particular, increase the viscosity. Typically, cellulose ethers are used as rheology modifiers for oil in water compositions and they are present in many oil in water compositions to enhance viscosity. They can be present in foaming compositions in order to increase viscosity. In most instances, their primary function is viscosity build up with some, very few, examples when they can function also as co-stabilizers of emulsions/foams due to sufficient hydrophobic character present in their structure.

Nanocrystalline cellulose is a crystalline portion of cellulose which can be formed by acid hydrolysis of cellulose combined with mechanical treatment. These nanometer size cellulose particles are crystalline in nature, insoluble in water, stable, chemically inactive and physiologically inert with attractive binding properties.

### BRIEF DESCRIPTION OF THE DRAWING

FIG. 1 is a graph showing viscosity vs. shear rate.

### DETAILED DESCRIPTION

Before explaining at least one embodiment of the inventive concept(s) in detail by way of exemplary drawings, experimentation, results, and laboratory procedures, it is to be understood that the inventive concept(s) is not limited in its application to the details of construction and the arrangement of the components set forth in the following description or illustrated in the drawings, experimentation and/or results. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed and claimed inventive concept(s) shall have the meanings that are commonly understood by those of ordinary skill in the art. Further, unless otherwise required by context, singular terms shall include pluralities and plural terms shall include the singular. Generally, nomenclatures utilized in connection with, and techniques of chemistry described herein are those well known and commonly used in the art. Reactions and purification techniques are performed according to manufacturer's specifications or as commonly accomplished in the art or as described herein. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analysis, pharmaceutical preparation, formulation, and delivery, and treatment of patients.

All patents, published patent applications, and non-patent publications mentioned in the specification are indicative of the level of skill of those skilled in the art to which this presently disclosed and claimed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the inventive concept(s) as defined by the appended claims.

As utilized in accordance with the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, and/or the variation that exists among the study subjects. The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

The presently disclosed and claimed inventive concept(s) is premised on the discovery that water soluble cellulose ethers in combination with nanocrystalline cellulose can act as stabilizers for multiphase systems, especially for aqueous multiphase systems. In particular, this combination can be used to stabilize solid particles dispersed in aqueous solutions, as well as oil in water emulsions. Further, it can be used to stabilize gas/liquid foam system.

The presently disclosed and claimed inventive concept(s) provides a stabilized multiphase composition comprising or consisting of or consisting essentially of a first phase, a second phase, cellulose ether and nanocrystalline cellulose. The cellulose ether can be water soluble non-ionic and/or anionic cellulose ether. The first phase can be a liquid. In one non-limiting embodiment, the liquid can be water. The second phase can be a gas, a solid or a liquid. In one non-limiting embodiment, the gas can be air, the solid can be water suspendable particles, and the liquid can be water insoluble organic liquid.

Cellulose is one of the most abundant biopolymers on earth, occurring in wood, cotton, hemp and other plant-based material and serving as the dominant reinforcing phase in plant structures. Cellulose can also be synthesized by algae, tunicates, and some bacteria. It is a homopolymer of glucose repeating units which are connected by 1-4 βglycosidic linkages. The 1-4 β-linkages form cellulose in linear chains, which interact strongly with each other through hydrogen bonds. Because of their regular structure and strong hydrogen bonds, cellulose polymers are highly crystalline and aggregate to form substructures and microfibrils. Microfibrils, in turn aggregate to form cellulosic fibers.

Purified cellulose from wood or agricultural biomass can be extensively disintegrated or produced by bacterial processes. If the cellulosic material is composed of nanosized fibers, and the properties of the material are determined by its nanofiber structure, these polymers are described as nanocelluloses. The terms are used interchangeably herein.

In general, nanocelluloses are rod shaped fibrils with a length/diameter ratio of approximately 20 to 200. In one non-limiting embodiment, the nanocelluloses have a diameter less than about 60 nm. In another non-limiting embodiment, the nanocelluloses have a diameter between about 4 nm to about 15 nm, and a length of about 150 nm to about 350 nm. The size and shape of the crystals vary with their origins. Nanocrystalline cellulose from wood is 3 to 5 nm in width and 20 to 200 nm in length. Other nanocrystalline cellulose obtained from other sources such as cotton may have slightly different dimensions.

The nanocellulose has high stiffness, large specific surface area, high aspect ratio, low density and reactive surfaces that can facilitate chemical grafting and modification. At the same time, the material is inert to many organic and inorganic substances.

The production of nanocellulose by fibrillation of cellulose fibers into nano-scale elements requires intensive mechanical treatment. However, depending upon the raw material and the degree of processing, chemical treatments may be applied prior to mechanical fibrillation. Generally preparation of nanocellulose can be described by two methods, acid hydrolysis and mechanical defibrillation. In the first method, nanocellulose can be prepared from the chemical pulp of wood or agricultural fiber mainly by acid hydrolysis to remove the amorphous regions, which then produces nano-size fibrils. The hydrolysis conditions are known to affect the properties of the resulting nanocrystals. Different acids also affect the suspension properties. Nanocrystal size, dimensions, and shape are also determined to a certain extent by the nature of the cellulose source.

The acid hydrolysis can be conducted using a strong acid under strictly controlled conditions of temperature, agitation and time. The nature of the acid and the acid-to-cellulosic ratio are also important parameters that affect the preparation of nanocellulose. Examples of the acids can include, but are not limited to, sulfuric acid, hydrochloric acid, phosphoric acid and hydrobromic acid. The hydrolysis temperature can range from room temperature up to about 70° C. and the corresponding hydrolysis time can be varied from about 30 minutes to about 12 hours depending on the temperatures. Immediately following hydrolysis, suspension can be diluted to stop the reaction.

In one non-limiting embodiment, the suspension can be diluted from about five-fold to about ten-fold to stop the reaction. Then the suspension can be centrifuged, washed once with water and re-centrifuged and washed again. This process can be repeated for about four to five times to reduce the acid content. Regenerated cellulose dialysis tubes or Spectrum Spectra/Pro regenerated cellulose dialysis membrane having a molecular cutoff of about 12,000-14,000 can be used to dialyze the suspension against distilled water for several days until the water pH reaches a constant value, for example but not by way of limiting, a pH value of about 7.0.

To further disperse and reduce the size of the cellulose crystals, the suspensions of cellulose crystals can be processed by either sonicating or passing through a high shear micro fluidizer. This kind of prepared material is referred to as nanocrystalline cellulose (NCC), cellulose nanocrystals, cellulose nanofibres or cellulose whiskers.

The second method is primarily a physical treatment. Bundles of microfibrils called cellulose microfibril or microfibrillated cellulose with diameters from tens of nanometers (nm) to micrometers (μm) are generated by using high pressure homogenizing and grinding treatments. A novel process using high-intensity ultrasonication has also been used to isolate fibrils from natural cellulose fibres. High intensity ultrasound can produce very strong mechanical oscillating power, so the separation of cellulose fibrils from biomass is possible by the action of hydrodynamic forces of ultrasound. This method produces a microfibrillated cellulose with a diameter less than about 60 nm, more preferably between about 4 nm to about 15 nm, and a length less than 1000 nm The microfibrillated cellulose can optionally further undergo chemical, enzymatic and/or mechanical treatment. Both methods for preparing nanocrystalline cellulose are described in U.S. Pat. No. 8,105,430, the entire disclosure of which is hereby incorporated by reference.

Nanocrystalline cellulose can be combined with cellulose ether to stabilize multiphase systems. Nonionic and anionic water soluble cellulose ethers are suitable for use in the presently disclosed and claimed inventive concept(s). The cellulose ethers can include, but are not limited to, methyl cellulose, methylhydroxypropyl cellulose, methyl hydroxyethyl cellulose, hydroxymethyl cellulose, hydroxyethyl cellulose, ethyl hydroxyethyl cellulose, carboxymethyl cellulose, cetyl hydroxyethylcellulose and combinations thereof. The combination can be added to multiphase systems directly to improve stability.

### Dispersed Particles

In order to stabilize particles dispersed in a liquid, especially in an aqueous medium, nanocrystalline cellulose and cellulose ether are blended together, preferably in de-ionized water. Generally, the ratio of cellulose ether to nanocrystalline cellulose can be in the range from about 10:1 to about 1:10 by weight. In one non-limiting embodiment, the ratio can be in the range from about 1:4 to about 1:4. In another non-limiting embodiment, the ratio can be in the range from about 4:1 to about 1:1.

The amounts of nanocrystalline cellulose and cellulose ether added to the aqueous system can be varied depending upon the concentration of the particles to be suspended in the liquid, as well as end-use requirements. Generally, from about 0.5 to about 5% of the combination by weight can be added to the aqueous formulation. Generally, such formulations may have about 1 to about 70% by weight insoluble particles.

Particles that are dispersed in aqueous systems generally have a relatively small particle size, for example but not by way of limitation, less than about 100 microns. The presently disclosed and claimed inventive concept(s) can be used to stabilize a wide variety of different particles such as coloring agents, for example but not by way of limitation, titanium dioxide, rutile, clay particles, water insoluble inorganic salts, zinc oxide, calcium carbonate, minerals, fillers, and the like.

One particular application for use in the presently disclosed and claimed inventive concept(s) is stabilization of water-based or latex paint formulations. These formulations typically are relatively viscous. The high viscosity helps to stabilize the pigments and particulate fillers. In many applications, the formulations are further diluted prior to use. This dilution step reduces the viscosity and thus the stability of the paint formulations. The presently disclosed and claimed inventive concept(s), as demonstrated below, provides a dispersion of “model” paint formulation, which is stable at normal concentrations and remains stable when further diluted down. Further on, the stability of this dispersion or “model” paint is not accompanied by an increase in medium-shear rate viscosity that would be the case when polymers are used alone in such compositions.

Water based coating compositions are formulated to achieve application and end coating properties. Coating components may include binders, pigments, extenders, polymers, surfactants, coalescents, neutralizing agents, water, etc. Pigments (e.g., titanium dioxide), inorganic colorants and extenders (e.g., clays and calcium carbonate) are granular solids incorporated into the coating formulation to contribute hiding, color, toughness, texture, and other properties. The binder is a film-forming latex polymer (e.g., acrylic, styrene acrylic, vinyl acrylic, vinyl acetate, etc.). As a liquid coating is applied and dries on a surface, this film-forming binder polymer serves to form a film (e.g., a dried coat) which bonds to the surface and also binds together all the non-volatile components of the paint including particularly any pigments and extenders present. The binder polymer imparts adhesion, gloss and is critical to durability, flexibility and toughness.

Rheology modifying polymers, dispersants, surfactants, foam control agents and coalescents are used to optimize the manufacturing process, “in-can stability”, application properties, surface wetting, flow and leveling properties, etc. There are also a variety of other additives added to coating formulations, such as, emulsifiers, adhesion promoters, UV stabilizers and biocides. Nanocrystalline cellulose in combination with cellulose ether can be used to stabilize the particulates dispersed in liquid paint formulations.

In order to test the stabilizing effect of nanocrystalline cellulose in combination with a cellulose ether, an aqueous dispersion of calcium carbonate particles was blended with an aqueous dispersion of hydroxyethyl cellulose (HEC) and nanocrystalline cellulose (weight ratio of about 2:1) to evaluate low shear rate viscosity and medium shear rate viscosity. The HEC used were Natrosol™ 250 HR/HHR, PC grade, which was available from Ashland Inc. The calcium carbonate used was MicroWhite 100, which had a particle size of about 38 to about 45 microns, and the aqueous formulation included about 0.2 wt % potassium tetra pyrophosphate. The particle size of pigments in paint was generally between as low as tens of nanometers and up to microns in aggregated state. The concentration of calcium carbonate was about 60%.

Initially, an aqueous viscous dispersion of HEC and NCC was formed by combining the two components together in deionized water. Calcium carbonate was then added to this viscous dispersion to form a mixture and the mixture was stirred with a spatula for about 3 to about 5 minutes. The formed composition was diluted with soft/deionized water in small increments to a desired level. Syneresis was evaluated after about four hours and hard packing/pigment settling was assessed after about 24 hours. In syneresis, one is looking for any water that has been expulsed from the dispersion or risen to the top of a container. Hard packing was tested by placing a wooden tongue depressor into the dispersion and stirred around. A positive hard packing result can be heavy residue on the wooden depressor.

A stable calcium carbonate dispersion with about 60% calcium carbonate, about 1% hydroxyethyl cellulose (Natrosol™ 250 HHR-P, available from Ashland Inc.) and no nanocrystalline was formed. When diluted 40% with deionized water, syneresis was observed after about 4 hours.

A similar dispersion as the above, again without nanocrystalline cellulose, was formed, and then diluted 11%. No syneresis or hard packing was observed. When diluted 28%, again no syneresis and no hard packing were observed. However, when diluted 40% by volume, syneresis was observed.

A mixture with about 1% hydroxyethyl cellulose (Natrosol™ 250HHR-P grade), and about 0.5% nanocrystalline cellulose, together with about 60% calcium carbonate was prepared and formed a stable dispersion. When the dispersion was diluted 40%, no syneresis and no hard packing were observed. In a similar dispersion with about 0.67% hydroxyethyl cellulose (Natrosol™ 250HHR-P grade) and about 0.33% nanocrystalline cellulose, about 40% dilution produced no syneresis, no hard packing and was stable.

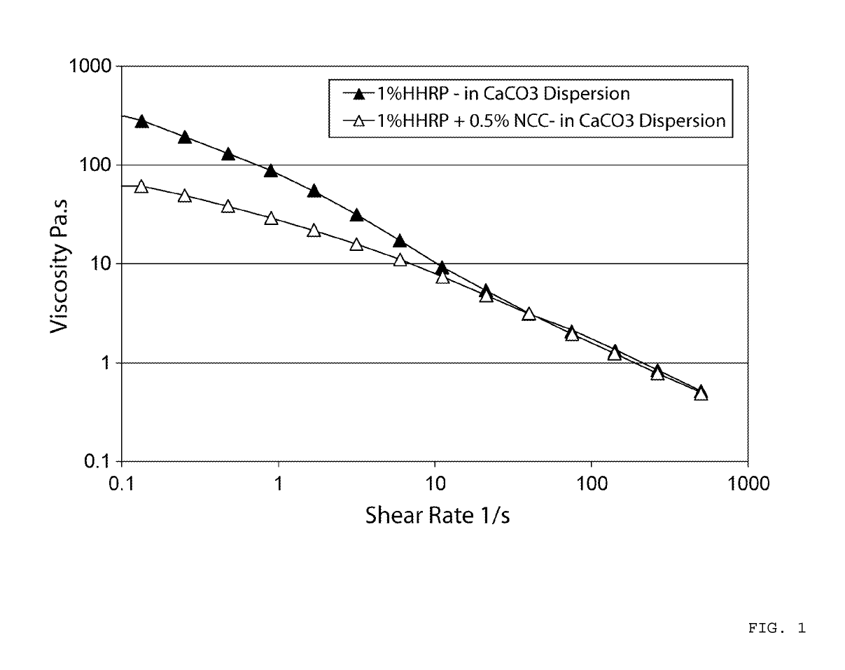

A dispersion of about 1% hydroxyethyl cellulose (Natrosol™ 250HHR-P grade) and about 0.5% nanocrystalline cellulose with about 60% calcium carbonate and water again diluted this time 58% produced a dispersion which exhibited no syneresis and no hard packing. In another example, a similar dispersion with about 0.67% hydroxyethylcellulose (Natrosol™ 250HR) and about 0.33% nanocrystalline cellulose, about 40% dilution produced no syneresis, no hard packing and was stable. FIG. 1 illustrates the boost in low shear viscosity of a calcium carbonate dispersion in water thickened with HEC-NCC and no change of medium shear rate viscosity was observed compared to HEC product without NCC.

These tests establish that cellulose ether/nanocrystalline cellulose blends can provide improved dilution tolerance of aqueous dispersions of insoluble particles and selectively increase only the low-shear rate viscosity, while not affecting the medium-shear rate viscosity. This permits the formation of dispersions and “model” paints based on cellulose ether/nanocrystalline cellulose, which can be further diluted without loss of stability and formulated without an undesired increase in medium shear rate viscosity or KU of paint.

### Oil in Water Emulsions

Nanocrystalline cellulose can also be combined with alkyl cellulose ether polymers to form stabilized emulsions of water insoluble organic liquids in water, for example but not by way of limitation, oil in water emulsions. Such emulsions may have less than about 50% organic liquid, generally about 1% to about 30%, and most commonly about 10% to about 30% and more typically about 20 to about 30% organic liquid or oil. This, of course, can vary depending on the end use requirement, as well as the particular organic liquid.

These emulsions can generally include from about 0.1 to about 20 percent of the alkyl cellulose ether by weight and about 0.01 to about 10 percent nanocrystalline cellulose by weight. In one non-limiting embodiment, the emulsions comprises from about 0.5% to about 5% of alkyl cellulose ether. Generally, the ratio of cellulose ether to nanocrystalline cellulose by weight can be from about 10:1 to about 1:10. In one non-limiting embodiment, the ratio can be from about 4:1 to about 1:4. In another non-limiting embodiment, the ratio can be from about 4:1 to about 1:1.

Generally, the alkyl cellulose ether can include, but are not limited to, ethyl hydroxyethyl cellulose, methylhydroxylethyl cellulose, carboxymethyl cellulose, cetyl hydroxyethyl cellulose, methyl hydroxylpropyl cellulose, and hydroxyethyl cellulose, as well as others.

Typically, the emulsions can include more than about 0.3% by weight nanocrystalline cellulose, more likely about 0.4% or greater, and, in particular about 0.5% nanocrystalline cellulose. In one non-limiting embodiment, the emulsion comprises from about 0.3% to about 1% nanocrystalline cellulose.

The NCC/alkyl cellulose ether blend can be used to stabilize a wide variety of oil in water emulsions including personal care products, household products, car care products and waxes, pesticides, herbicides and other industrial emulsions.

In typical oil in water emulsions for personal care products, surfactants, and in particular anionic surfactants, are added to stabilize the emulsions. However, the use of nanocrystalline cellulose can permit one to reduce the amounts of surfactants present and, in many cases, to eliminate anionic surfactants. Preferably, the composition can include less than about 1% anionic surfactant and in a preferred case essentially no surfactant.

Other emulsifiers can be used in combination with the NCC/alkyl cellulose ether blend such as non-ionic emulsifiers, as well as others. Examples of non-ionic emulsifiers can include, but are not limited to, fatty alcohols, Steareth-n and Ceteareth-n.

The composition is a personal care product when it contains at least one active personal care active ingredient or benefiting agent. The personal care active ingredients or benefiting agents can include, but are not limited to, analgesics, anesthetics, antibiotic agents, antifungal agents, antiseptic agents, antidandruff agents, antibacterial agents, vitamins, hormones, anti-diarrhea agents, corticosteroids, anti-inflammatory agents, vasodilators, karyolitic agents, dry-eye compositions, wound-healing agents, anti-infection agents, UV absorbers, moisturizers, humectants, emolliency, lubricating, softening, hair-detangling, hair relaxers, hair sculpturing, hair removing, dead-skin removing, as well as solvents, diluents, adjuvants and other ingredients such as water, ethyl alcohol, isopropyl alcohol, propylene glycol, higher alcohols, glycerin, sorbitol, mineral oil, preservatives, surfactants, propellants, fragrances, essential oils, and viscosifying agents. In such compositions, oils, such as mineral oils, vegetable derived oils, esters, synthetic waxes, and silicones are typically used.

Personal care compositions can include hair care, skin care, sun care, nail care, and oral care compositions. Examples of personal care active ingredients or benefiting agents in the personal care products according to the present invention can include, but are not limited to,
1) Perfumes, which give rise to an olfactory response in the form of a fragrance, and deodorant perfumes which in addition to providing a fragrance response can also reduce body malodor;
2) Skin coolants, such as menthol, menthyl acetate, menthyl pyrrolidone carboxylate N-ethyl-menthane-3-carboxamide and other derivatives of menthol, which give rise to a tactile response in the form of a cooling sensation on the skin;
3) Emollients, such as isopropylmyristate, silicone materials, mineral oils and vegetable oils which give rise to a tactile response in the form of an increase in skin lubricity;
4) Deodorants other than perfumes, whose function is to reduce the level of or eliminate micro flora at the skin surface, especially those responsible for the development of body malodor. Precursors of deodorants other than perfumes can also be used;
5) Antiperspirant actives, whose function is to reduce or eliminate the appearance of perspiration at the skin surface;
6) Moisturizing agents, which keep the skin moist by either adding moisture or preventing moisture from evaporating from the skin;
7) Sunscreen active ingredients that protect the skin and hair from UV and other harmful light rays from the sun. In accordance with this presently disclosed and claimed inventive concept(s) a therapeutically effective amount can normally be from about 0.01 to about 10% by weight, preferably about 0.1 to about 5% by weight of the composition;
8) Hair treatment agents, which condition the hair, cleanse the hair, detangles hair, act as styling agents, volumizing and gloss agents, color retention agents, anti-dandruff agents, hair growth promoters, hair dyes and pigments, hair perfumes, hair relaxers, hair bleaching agents, hair moisturizers, hair oil treatment agents, and antifrizzing agents; and
9) Oral care agents, such as toothpastes, dentifrices and mouth washes, which can clean, whiten, deodorize and protect the teeth and gum.

In accordance with the presently disclosed and claimed inventive concept(s), the composition may be used in a household care composition. The household care composition additionally comprises water and at least one household active care ingredient or benefiting agent. The household care active ingredient or benefiting agent must provide some benefit to the user. Examples of active ingredients or benefiting agents that may suitably be included, but not limited to,
1) Perfumes, which give rise to an olfactory response in the form of a fragrance and deodorant perfumes which in addition to providing a fragrance response can also reduce odor;
2) Insect repellent agents, whose function is to keep insects from a particular area or attacking skin;
3) Pet deodorizers or insecticides such as pyrethrins that reduce pet odor;
4) Pet shampoo agents and actives, whose function is to remove dirt, foreign material and germs from the skin and hair surfaces;
5) Industrial grade bar, shower gel, and liquid soap actives that remove germs, dirt, grease and oil from skin, sanitize skin, and condition the skin;
6) Disinfecting ingredients that kill or prevent growth of germs in a house or public facility;
7) A laundry softener active, which reduces static and makes fabric feel softer;
8) Laundry or detergent or fabric softener ingredients that reduce color loss during wash, rinse, and drying cycle of fabric care;
9) Toilet bowl cleaning agents, which remove stains, kill germs, and deodorize; and
10) Fabric sizing agent which enhances appearance of the fabric.

The above lists of personal care and household care active ingredients or benefiting agents are only examples and are not a complete list of active ingredients or benefiting agents that can be used. Other ingredients that are used in these types of products are well known in the industry. In addition to the above ingredients conventionally used, the composition according to the presently disclosed and claimed inventive concept(s) can optionally include ingredients such as colorants, preservatives, antioxidants, nutritional supplements, alpha or beta hydroxyl acids, activity enhancers, emulsifiers, functional polymers, alcohols having 1-6 carbons, fats or fatty compounds, antimicrobial compounds, zinc, pyrithione, silicone material, hydrocarbon polymers, emollients, oils, surfactants, medicaments, flavors, fragrances, suspending agents, and mixtures thereof.

In order to test the ability of cellulose ether and nanocrystalline cellulose to stabilize oil in water emulsions, oil in water emulsions were formed from about 10% white mineral oil, 1% preservative, 0.9% hydrophobically modified hydroxyethyl cellulose (Natrosol™ Plus 330 CS, available from Ashland Inc.), and variable amounts of nanocrystalline cellulose from about 0.1% to about 0.5%. The remainder was water. At concentrations of about 0.1% and about 0.2% nanocrystalline cellulose, the oil in water emulsion was relatively unstable. At concentrations of about 0.3% nanocrystalline cellulose, the stability was increased. At about 0.4% and about 0.5% nanocrystalline cellulose, the emulsions were stable. These emulsions were homogeneous with a lotion-like appearance with no off-color or pH change upon aging. These emulsions had been remained stable for at least about four weeks at temperatures about 45° C., and were easy to apply on skin and provided a non-greasy skin feel. Oil in water emulsions made with about 1.5% and about 2% hydrophobically modified hydroxyethyl cellulose without nanocrystalline cellulose was not stable.

### Foams

Nanocrystalline cellulose in combination with water soluble cellulose ethers can be used to stabilize foams. The cellulose ether/nanocrystalline cellulose blend can be dispersed in deionized water and blended with a foamable aqueous liquid.

The foamable aqueous liquid can include water, and a composition which can generate foam. There are a wide variety of different compositions that can generate foam, including oils, fats, fatty acids, surfactants, proteins, as well as many others. Any foam generating agent which is compatible with the nanocrystalline cellulose can be used in the presently disclosed and claimed inventive concept(s).

The foam compositions can include a wide variety of different consumer and/or industrial products. These can include, for example but by no way of limitation, food products such as whip cream; whip cream substitutes; shampoos both for human and pet use; and soaps for car washing and other applications.

Generally, the concentration of NCC and cellulose ether can vary depending upon the end use applications. Concentrations of the cellulose ether of about 0.25%-1.0% by weight can be employed in the foaming composition of the presently disclosed and claimed inventive concept(s). In one non-limiting embodiment, the concentration of the cellulose ether can be from about 0.5 to about 0.75%.

Generally, the concentration of nanocrystalline cellulose can be from about 0.1 to about 1%. In one non-limiting embodiment, the concentration of the nanocrystalline cellulose can be and from about 0.25% to about 0.5%.

In order to test the foam stabilization of the nanocrystalline cellulose/cellulose ether blend, an exemplary foam-forming solution was prepared containing about 12% sodium laureth sulfate with about 2% cocamide propyl betaine, which was subsequently diluted to a 1% total actives solution in deionized water. This was used as a liquid medium to prepare dispersions of hydroxyethyl cellulose, (a medium viscosity hydroxyethyl cellulose commercially available from Ashland Inc. as Natrosol™ 250 HR), nanocrystalline cellulose and their blends with various concentrations of the components.

The tested solutions/dispersions were whipped with a Waring Blender to create foam. Immediately, the foam was poured in a funnel over 20 mesh screen. The time required to drain was recorded. As shown in Table 1, the test solution without cellulose ether or nanocrystalline cellulose produced a stable foam which lasted about 37 seconds, whereas with nanocrystalline cellulose by itself or the cellulose ether by itself (both tested at 0.75% in water), the stability was reduced. Stability improved relative to cellulose ether by itself or nanocrystalline cellulose by itself, when 0.25% of the cellulose ether was combined with 0.25% of the nanocrystalline cellulose. Although the time was 15 seconds, it is believed that the charge of the surfactant reduced the stability or destroyed the stability of the nanocrystalline cellulose. However, even with the anionic surfactant used in the test solution, and at 0.5% cellulose ether and 0.5% nanocrystalline cellulose, the foam stability was recorded at over 2 minutes.

These results demonstrate that the combination of water soluble cellulose ether with nanocrystalline cellulose can stabilize a variety of different multiphase systems, either particulate/water systems, as in the case of, for example but not by way of limitation, water based dispersions/“model” paints, oil in water emulsions such as shampoos, detergents and the like, and foams as in the case of a wide variety of different products such as whip cream substitute products, shaving cream, detergents, formed with or without oil.

It is, of course, not possible to describe every conceivable combination of the components or methodologies for purpose of describing the disclosed information, but one of ordinary skill in the art can recognize that many further combinations and permutations of the disclosed information are possible. Accordingly, the disclosed information is intended to embrace all such alternations, modifications and variations that fall within the spirit and scope of the appended claims.

TABLE 1

| | System Data | | | | | | | | |
|---|---|---|---|---|---|---|---|---|---|
| | 0.5% | 0.5% | 0.5% | 0.5% | 0.25% | 0.25% | 0.75% | 0.75% | 1% (SLES/ |
| | 250HR + | 250HR + | 250HR + | 250HR + | 250HR | 250HR + | NCC − | 250HR − | CAPB = |
| | 0.5% NCC | 0.25% NCC | 0.17% NCC | 0.1% NCC | 0.25% NCC | 0.1% NCC | Control | Control | 12%/2%) |
| | Time (seconds in funnel) | | | | | | | | |
| | >2 min | sec | 15 sec | 12 sec | 15 sec | 10 sec. | <5 sec | 15 sec | 37 sec |
| Visual | Thick | Thick | Thin Foam | Thin Foam | Semi Thick | Thin Foam | No Foam | Some foam | Thick |
| Observation | Opaque | Opaque | Opaque | Opaque | Opaque | Opaque | generated | generate but | Opaque |
| | Creamy | Passed | Small | Small | Medium size | Small | Viscosity very | foam quickly | Passed |
| | Did not pass | through mesh | bubbles | bubbles | bubbles | bubbles | low | dissipated | through mesh |
| | through | Large bubbles | remain | remain | remain after | remain | passed through | | Bubbles remain |
| | mesh | remain after | | | blending | | mesh very | | after blending |
| | | blending | | | | | quickly | | |

What is claimed is:

1. A stabilized multiphase composition comprising a first phase, a second phase, water soluble non-ionic cellulose ether and nanocrystalline cellulose.

2. The stabilized multiphase composition of claim 1, wherein the first phase comprises water.

3. The stabilized multiphase composition of claim 1, wherein the second phase is selected from the group consisting of gas, water suspendable particles, water insoluble organic liquids, and combinations thereof.

4. The stabilized multiphase composition of claim 2, wherein the second phase comprises oil.

5. The stabilized multiphase composition of claim 4, wherein the multiphase composition is surfactant free.

6. The stabilized multiphase composition of claim 4, further comprising a surfactant.

7. The stabilized multiphase composition of claim 4, wherein the multiphase composition comprises from about 0.1% to about 20% alkyl cellulose ether.

8. The stabilized multiphase composition of claim 7, wherein the multiphase composition comprises from about 0.3% to about 1% nanocrystalline cellulose.

9. The stabilized multiphase composition of claim 4, wherein the cellulose ether is selected from the group consisting of methyl hydroxyethyl cellulose, carboxymethyl cellulose, ethyl hydroxyethyl cellulose, cetyl hydroxyethyl cellulose, methylhydroxypropyl cellulose and hydroxyethyl cellulose.

10. The stabilized multiphase composition of claim 4, wherein the multiphase composition comprises from about 1% to about 30% of oil.

11. The stabilized multiphase composition of claim 4, wherein the multiphase composition is a personal care product.

12. The stabilized multiphase composition of claim 11, wherein the personal care product further comprises a personal care active compound.

13. The stabilized multiphase composition of claim 12, wherein the personal care product is shampoo.

14. The stabilized multiphase composition of claim 4, wherein the multiphase is a food product.

15. The stabilized multiphase composition of claim 4, wherein the oil is selected from the group consisting of mineral oils, vegetable derived and synthetic waxes, esters, and silicones.

16. The stabilized multiphase composition of claim 2, wherein the second phase comprises insoluble particles.

17. The stabilized multiphase composition of claim 16, wherein the insoluble particles are selected from the group selected from pigments, inert fillers, and coloring agents.

18. The stabilized multiphase composition of claim 16, wherein the insoluble particles are selected from the group consisting of titanium dioxide, calcium carbonate, rutile, clay and zinc oxide.

19. The stabilized multiphase composition of claim 16, wherein the cellulose ether is selected from the group consisting of hydroxyethyl cellulose, methyl hydroxyethyl cellulose, ethyl hydroxylethyl cellulose, methyl hydroxypropyl cellulose, and cetyl hydroxyethyl cellulose.

20. The stabilized multiphase composition of claim 16, further comprising a film forming latex polymer.

21. The stabilized multiphase composition of claim 16, wherein the multiphase composition is paint.

22. A method of stabilizing insoluble particulate material in water comprising combining a cellulose ether with nanocrystalline cellulose to form a blend, adding the blend to the water to form a dispersion and mixing the insoluble particulate material in the dispersion.

23. The stabilized multiphase composition of claim 1, wherein the second phase is a gas.

24. The stabilized multiphase composition of claim 23, further comprising a foam generating agent.

25. The stabilized multiphase composition of claim 23, wherein the gas is air.

26. The stabilized multiphase composition of claim 23, wherein the multiphase composition is a personal care product.

27. The stabilized multiphase composition of claim 23, wherein the first phase comprises oil.

28. The stabilized multiphase composition of claim 23, wherein the multiphase composition is a food product.
